(19) United States
(12) Patent Application Publication
(10) Pub. No.: US 2012/0088918 A1
(43) Pub. Date: Apr. 12, 2012

(54) Continuous Method For Producing Esters Of Aromatic Carboxylic Acids

(75) Inventors: Matthias Krull, Harxheim (DE); Roman Morschhaeuser, Mainz (DE)

(73) Assignee: CLARIANT FINANCE (BVI) LIMITED, Tortola (VG)

(21) Appl. No.: 13/378,167

(22) PCT Filed: Jun. 9, 2010
(86) PCT No.: PCT/EP2010/003447

§ 371 Date: Dec. 14, 2011

(30) Foreign Application Priority Data
Jun. 30, 2009 (DE) 10 2009 031 054.1

(51) Int. Cl. C07D 213/803; C07C 67/08

(52) U.S. Cl. 546/327; 560/103; 560/67; 560/85

(57) ABSTRACT

The invention relates to a continuous method for producing aromatic carboxylic acid esters by reacting at least one aromatic carboxylic acid of formula (I) Ar—COOH (I), wherein Ar represents an optionally substituted aryl group with 5 to 50 atoms, with at least one alcohol of formula (II) R²—(OH)ₙ (II), wherein R² represents an optionally substituted hydrocarbon group with 1 to 100 C atoms and n is an integer from 1 to 10, in the presence of at least one transesterification catalyst in a reaction tube, the longitudinal axis of which extends in the direction of propagation of the microwaves of a monomode microwave applicator, under microwave irradiation to form the ester.

The present invention relates to a continuous method for producing esters of aromatic carboxylic acids under microwave irradiation on an industrial scale.

Esters are an industrially very important substance group which is used widely and is used, for example, as plasticizer, lubricant and also as a constituent of cosmetics and pharmaceuticals. A proven and often used method for producing esters is the condensation of carboxylic acids with alcohols in the presence of catalysts. In the process, the reaction mixture is usually heated for several hours and the water that is formed is removed. Methods are also known in which the esterification is carried out in a closed system under pressure and high temperatures. For example, WO 2007/126166 discloses a conventionally thermal esterification of fatty acids with alcohols at temperatures of from 200 to 350° C. and pressures of up to 10 bar. During the reaction over several hours, in the course of which the water of reaction which is formed is continuously removed with excess alcohol, however, only an incomplete conversion to the methyl ester is achieved, meaning that a complex work-up and/or further processing of the crude product is required. Another problem of such high-temperature reactions is the corrosivity of the reaction mixtures which, on the one hand, leads to damage of the reaction vessels and, on the other hand, to undesired metal contents in the esters produced in this way.

A more recent approach to the synthesis of esters is the microwave-supported reaction of carboxylic acids and alcohols, as a result of which especially the reaction times required for satisfactory yields are considerably reduced.

Q. Yang et al. (Synth. Commun. 2008, 38, 4107-4115) describe acid-catalyzed esterifications of various carboxylic acids with alcohols under microwave irradiation. The reactions are carried out at 100° C. on a laboratory scale and lead to high conversions.

US 2005/0274065 discloses a method in which fatty acids are esterified with alcohols in the presence of catalysts and/or under the influence of microwave energy. Here, in one specific embodiment, the reaction material located in a receiver is continually circulated and, in so doing, passed through a stirred container located in a microwave applicator. Only following repeated conveyance through the microwave applicator are high degrees of esterification achieved.

The scale-up of such microwave-supported reactions from the laboratory to an industrial scale and thus the development of plants which are suitable for a production of several tons, for example several tens, several hundreds or several thousands of tons, per year with space-time yields of interest for industrial-scale applications has, however, not been realized to date. One reason for this is the penetration depth of microwaves into the reaction material, which is usually limited to a few millimeters to a few centimeters, which limits especially reactions carried out in batch processes to small vessels, or leads to very long reaction times in stirred reactors. Tight limits are placed on an increase in the field strength, which is desirable for the irradiation of large amounts of substance with microwaves, especially in the multimode devices used preferentially to date for scale-up of chemical reactions as a result of the discharge processes and plasma formation which then arise. Furthermore, the inhomogeneity of the microwave field, which leads to local overheating of the reaction material in multimode microwave devices and is caused by more or less uncontrolled reflections of the microwaves injected into the microwave oven at the walls thereof and the reaction mixture, presents problems in the scale-up. Furthermore, the microwave absorption coefficient of the reaction mixture, which often changes during the reaction, presents difficulties with regard to a safe and reproducible reaction regime.

WO 90/03840 discloses a continuous method for carrying out various chemical reactions, such as, for example, esterifications, in a continuous laboratory microwave reactor. However, the achieved yields and also the reaction volume of 24 ml of the microwave operated in multimode, do not permit upscaling to the industrial sector. The efficiency of this method with regard to the microwave absorption of the reaction material is low on account of the microwave energy being more or less homogeneously distributed over the applicator space in multimode microwave applicators and not focused on the tube coil. A significant increase in the microwave power injected can lead to undesired plasma discharges or to so-called thermal runaway effects. Furthermore, the spatial inhomogeneities of the microwave field in the applicator space, which are referred to as hot-spots and change over time, make a safe and reproducible reaction regime on a large scale impossible.

Also known are monomode or single-mode microwave applicators which use a single wave mode which propagates in only one three-dimensional direction and is focused onto the reaction vessel by waveguides of exact dimensions. Although these instruments do allow relatively high local field strengths, on account of the geometric requirements (e.g. the intensity of the electrical field is at its greatest at its wave crests and approaches zero at the nodes), they have hitherto been restricted to small reaction volumes (≦50 ml) on the laboratory scale.

For example, Chemat et al. (J. Microwave Power and Electromagnetic Energy 1998, 33, 88-94) describe various continuous esterifications in a monomode microwave cavity, where the microwave guide is perpendicular to the reaction tube. Here, accelerated conversions are observed in the case of heterogeneously catalyzed esterifications. The volume of only 20 ml available for the microwave irradiation, however, requires that the reactants be repeatedly conveyed through the irradiation zone in order to achieve interesting yields. A significant increase in the cross section of the reaction tube is not possible on account of the geometry of the applicator and is also not suitable for the upscaling on account of the low penetration depth of microwaves.

Pipus et al. (First European Congress on Chemical Engineering, Firenze, Italy, May 4-7, 1997; AIDIC: Milan, Italy, 1997; pp. 45-48) disclose homogeneously and also heterogeneously catalyzed esterifications of benzoic acid with ethanol in a continuous tubular reactor heated with microwave radiation. At a pressure of 7 atm and a temperature of 140° C., with a residence time in the reactor of 127 seconds, a conversion of 30% is achieved.

Wilson et al. (Org. Process Res. Dev. 2004, 8, 535-538) disclose a continuous microwave reactor in which a mixture of 2,4,6-trimethylbenzoic acid and methanol is passed in a glass coil with a volume of 4 ml through a monomodal microwave field and is thereby esterified. On account of the geometric conditions of this set-up, the method cannot be transferred to large-scale applications.

A method was therefore sought for producing esters of aromatic carboxylic acids, in which aromatic carboxylic acid and alcohol can also be converted to the ester on an industrial scale under microwave irradiation. In this connection, the aim was to achieve the highest possible, i.e. up to quantitative, conversion rates and yields. Furthermore, the method should permit as energy-saving a production of the esters as possible, i.e. the microwave power used should be absorbed as quantitatively as possible by the reaction material and the method should thus offer a high energetic efficiency. In the process, only minor amounts of by-products, if any, should be produced. The esters should also have the lowest possible metal content and a low intrinsic coloration. Moreover, the method should ensure a safe and reproducible reaction regime.

Surprisingly, it has been found that esters of aromatic carboxylic acids can be produced in industrially relevant amounts by direct reaction of aromatic carboxylic acids with alcohols in a continuous method by only briefly heating by means of irradiation with microwaves in a reaction tube, the longitudinal axis of which is in the direction of propagation of the microwaves of a monomode microwave applicator. Here, the microwave energy injected into the microwave applicator is virtually quantitatively absorbed by the reaction material. The method according to the invention additionally has high safety during the procedure and offers high reproducibility of the reaction conditions established. The esters produced by the method according to the invention exhibit a high purity and low intrinsic coloration not obtainable in comparison to by conventional production methods without additional method steps.

The invention provides a continuous method for producing carboxylic esters, in which at least one aromatic carboxylic acid of the formula (I)

Ar—COOH (I)

in which Ar is an optionally substituted aryl radical having 5 to 50 atoms, is reacted with at least one alcohol of the formula (II)

R²—(OH)ₙ (II)

in which
R² is an optionally substituted hydrocarbon radical having 1 to 100 carbon atoms and
n is a number from 1 to 10,
   in the presence of at least one esterification catalyst with microwave irradiation in a reaction tube, the longitudinal axis of which extends in the direction of propagation of the microwaves of a monomode microwave applicator, to give the ester.

Ar is preferably an aryl radical which carries at least one carboxyl group bonded to an aromatic system. Aromatic systems are understood as meaning cyclic, through-conjugated systems with (4 n+2) π electrons, in which n is a natural integer and preferably 1, 2, 3, 4 or 5. The aromatic system can be mono- or polycyclic, such as, for example, di- or tricyclic. Preferably, the aromatic system has 6 to 25 atoms, particularly preferably 6 to 18 atoms. The aromatic system is preferably formed from carbon atoms. In a further preferred embodiment, besides carbon atoms, it comprises one or more heteroatoms such as, for example, nitrogen, oxygen and/or sulfur. Examples of such aromatic systems are benzene, naphthalene, indole, phenanthrene, pyridine, furan, pyrrole, thiophene and thiazole. Besides the carboxyl group, the aromatic system can carry one or more, such as, for example, one, two, three or more, identical or different further substituents. Suitable further substituents are, for example, alkyl, alkenyl and halogenated alkyl radicals, hydroxy, hydroxyalkyl, alkoxy, poly(alkoxy), halogen, carboxyl, amide, cyano, nitrile and/or nitro groups. These substituents can be bonded at any desired position of the aromatic system. However, the aryl radical carries at most as many substituents as it has valences.

In a specific embodiment, the aryl radical Ar of the formula (I) carries further carboxyl groups. Thus, the method according to the invention is likewise suitable for reacting aromatic carboxylic acids having, for example, two or more carboxyl groups. The degree of esterification can be controlled for example via the stoichiometry between acid (I) and alcohol (II) in the reaction mixture.

Of particular suitability is the method according to the invention for the esterification of alkylarylcarboxylic acids such as, for example, alkylphenylcarboxylic acids. These are aromatic carboxylic acids in which the aryl radical Ar carrying the carboxyl group additionally carries at least one alkyl or alkenyl radical. The method in the case of the esterification of alkylbenzoic acids which carry at least one alkyl radical having 1 to 50 carbon atoms, preferably having 2 to 20 carbon atoms and in particular 1 to 12 carbon atoms, such as, for example, 1 to 4 carbon atoms, is particularly advantageous.

Furthermore, of particular suitability is the method according to the invention for the esterification of aromatic carboxylic acids, the aryl radical Ar of which carries one or more, such as, for example, two, three or more, hydroxyl groups and/or hydroxyalkyl groups. In the case of the esterification especially with at least equimolar amounts of alcohol of the formula (II), an esterification of the carboxyl group selectively takes place here; no esterification of the phenolic OH group with the formation of polyesters takes place.

Suitable aromatic carboxylic acids are, for example, benzoic acid, phthalic acid, isophthalic acid, terephthalic acid, the various isomers of naphthalenecarboxylic acid, of pyridinecarboxylic acid and of naphthalenedicarboxylic acid, and also trimellitic acid, trimesic acid, pyromellitic acid and mellitic acid, the various isomers of methoxybenzoic acid, hydroxybenzoic acid, hydroxymethylbenzoic acid, hydroxymethoxybenzoic acid, hydroxydimethoxybenzoic acid, hydroxyisophthalic acid, hydroxynaphthalenecarboxylic acid, hydroxypyridinecarboxylic acid and hydroxymethylpyridinecarboxylic acid, hydroxyquinolinecarboxylic acid and o-toluic acid, m-toluic acid, p-toluic acid, o-ethylbenzoic acid, m-ethylbenzoic acid, p-ethylbenzoic acid, o-propylbenzoic acid, m-propylbenzoic acid, p-propylbenzoic acid, 3,4-dimethylbenzoic acid and thiophenecarboxylic acid. Mixtures of different aryl- and/or alkylarylcarboxylic acids are likewise suitable.

In a preferred embodiment, R² is an aliphatic radical. This has preferably 1 to 24, particularly preferably 2 to 18 and specifically 3 to 6, carbon atoms. The aliphatic radical can be linear, branched or cyclic. It can also be saturated or unsaturated, it is preferably saturated. The hydrocarbon radical can carry substituents such as, for example, halogen atoms, halogenated alkyl radicals, hydroxyl, C₁-C₅-alkoxyalkyl, cyano, nitrile, nitro and/or C₅-C₂₀-aryl groups, such as, for example, phenyl radicals. The C₅-C₂₀-aryl radicals can for their part be optionally substituted with halogen atoms, halogenated alkyl radicals, hydroxyl, C₁-C₂₀-alkyl, C₂-C₂₀-alkenyl, C₁-C₅-alkoxy groups, such as, for example, methoxy, ester, amide, cyano, nitrile and/or nitro groups. Particularly preferred aliphatic radicals are methyl, ethyl, n-propyl, isopropyl, n-butyl, isobutyl and tert-butyl, n-hexyl, cyclohexyl, n-octyl, n-decyl, n-dodecyl, tridecyl, isotridecyl, tetradecyl, hexadecyl, octadecyl and methylphenyl.

In a further preferred embodiment, R² is an optionally substituted C₆-C₁₂-aryl group or an optionally substituted heteroaromatic group having 5 to 12 ring members. Preferred heteroatoms are oxygen, nitrogen and sulfur. Further rings can be fused onto the C₆-C₁₂-aryl group or the heteroaromatic group having 5 to 12 ring members. The aryl or heteroaromatic group can thus be mono- or polycyclic. Examples of suitable substituents are halogen atoms, halogenated alkyl radicals and also alkyl, alkenyl, hydroxy, hydroxyalkyl, alkoxy, ester, amide, nitrile and nitro groups.

In a specific embodiment, the radical R² carries one or more, such as, for example, two, three, four, five, six or more, further hydroxyl groups, but not more hydroxyl groups than the radical R² has carbon atoms or than the aryl group has valences. The hydroxyl groups can be bonded to adjacent carbon atoms or else to further removed carbon atoms of the hydrocarbon radical, but at most one OH group per carbon atom. Thus, the method according to the invention is also suitable for the esterification of polyols such as, for example, ethylene glycol, 1,2-propanediol, 1,3-propanediol, neopentyl glycol, glycerol, sorbitol, pentaerythritol, fructose and glucose. The esterification can be conducted here to full esters or else partial esters. The degree of esterification can be controlled here for example via the stoichiometry between carboxylic acid and alcohol in the reaction mixture.

In cases where the carboxylic acid (I) contains two or more carboxyl groups and the alcohol (II) contains two or more hydroxyl groups and/or both reactants in each case carry at least one carboxyl group and at least one hydroxyl group, oligomers and polymers can also be produced by the method according to the invention. In the case of such polycondensations, the viscosity of the reaction mixture, which increases during the microwave irradiation, is to be taken into consideration when designing the apparatus.

In a further preferred embodiment, R² is an alkyl radical interrupted with heteroatoms. Particularly preferred heteroatoms are oxygen and nitrogen. If the radical R² contains nitrogen atoms, then these nitrogen atoms carry no acidic protons though.

Thus, R² is preferably radicals of the formula (III)

—(R⁴—O)ₙ—R⁵ (III)

in which
R⁴ is an alkylene group having 2 to 18 carbon atoms, preferably having 2 to 12 and in particular 2 to 4, carbon atoms, such as, for example, ethylene, propylene, butylene or mixtures thereof,
R⁵ is hydrogen or a hydrocarbon radical having 1 to 24 carbon atoms or a group of the formula —R⁴—NR¹⁰R¹¹,
n is a number between 1 and 500, preferably between 2 and 200 and in particular between 3 and 50, such as, for example, between 4 and 20, and
R¹⁰, R¹¹ independently of one another, are an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, where the polyoxyalkylene units are derived from alkylene oxide units having 2 to 6 carbon atoms, or R¹⁰ and R¹¹ together with the nitrogen atom to which they are bonded are a ring having 4, 5, 6 or more ring members.

Examples of suitable alcohols are methanol, ethanol, n-propanol, isopropanol, n-butanol, isobutanol, tert-butanol, pentanol, neopentanol, n-hexanol, isohexanol, cyclohexanol, heptanol, octanol, decanol, dodecanol, tetradecanol, hexadecanol, octadecanol, eicosanol, ethylene glycol, 2-methoxyethanol, propylene glycol, diethylene glycol, triethylene glycol, polyethylene glycol, polypropylene glycol, triethanolamine, N,N-dimethylethanolamine, N,N-diethylethanolamine, phenol, naphthol and mixtures thereof. Also of suitability are fatty alcohol mixtures obtained from natural raw materials, such as, for example, coconut fatty alcohol, palm kernel fatty alcohol and tallow fatty alcohol.

The method is particularly suitable for producing methyl benzoate, ethyl benzoate, n-hexyl benzoate, o-tolyl benzoate, methyl p-toluate, methyl p-hydroxybenzoate, ethyl p-hydroxybenzoate, methyl nicotinate, ethyl nicotinate, phenyl nicotinate.

If the carboxylic acid (I) contains two or more carboxyl groups and the alcohol (II) contains two or more hydroxyl groups, it is also possible to produce polymers by the method according to the invention. In this case, the viscosity of the reaction mixture, which increases during the microwave irradiation, should be taken into consideration when designing the apparatus.

In the method according to the invention, aromatic carboxylic acid (I) and alcohol (II) can be reacted with one another in any desired ratios. Preferably, the reaction between carboxylic acid and alcohol takes place with molar ratios of from 20:1 to 1:20, preferably from 10:1 to 1:10 and specifically from 3:1 to 1:3, such as, for example, from 1.5:1 to 1:1.5, in each case based on the mole equivalents of carboxyl groups and hydroxyl groups. In a specific embodiment, carboxylic acid and alcohol are used in equimolar amounts. If the aromatic carboxylic acid (I) carries one or more hydroxyl groups, the reaction preferably takes place with at least equimolar fractions of alcohol (II), particularly preferably in the ratio of aromatic carboxylic acid (I) to alcohol (II) of from 1:1.01 to 1:50, specifically in the ratio 1:1.5 to 1:20, such as, for example, 1:2 to 1:10.

In many cases, it has proven to be advantageous to work with an excess of alcohol, i.e. molar ratios of hydroxyl groups to carboxyl groups of at least 1.01:1.00 and in particular between 50:1 and 1.02:1, such as, for example, between 10:1 and 1.1:1. Here, the carboxyl groups are converted virtually quantitatively to the ester. This method is particularly advantageous if the alcohol used is readily volatile. Readily volatile means here that the alcohol has a boiling point at atmospheric pressure of preferably below 200° C. and particularly preferably below 160° C., such as, for example, below 100° C., and can thus be separated off from the ester by distillation.

The esterifications are carried out in the method according to the invention in the presence of homogeneous catalysts, heterogeneous catalysts or mixtures thereof. Both acidic and alkali catalysts are suitable here. Esterification catalysts preferred according to the invention are acidic inorganic, organometallic or organic catalysts and mixtures of two or more of these catalysts.

Acidic inorganic catalysts within the context of the present invention include, for example, sulfuric acid, phosphoric acid, phosphonic acid, hypophosphorous acid, aluminum sulfate hydrate, alum, acidic silica gel and acidic aluminum hydroxide. It is also possible to use, for example, aluminum compounds of the general formula Al(OR¹⁵)₃ and titanates of the general formula Ti(OR¹⁵)₄ as acidic inorganic catalysts, where the radicals R¹⁵ can in each case be identical or different and, independently of one another, are selected from C₁-C₁₀-alkyl radicals, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, sec-hexyl, n-heptyl, n-octyl, 2-ethylhexy, n-nonyl or n-decyl, C₃-C₁₂-cycloalkyl radicals, for example cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl. The radicals R¹⁵ in Al(OR¹⁵)₃ or Ti(OR¹⁵)₄ are preferably in each case identical and selected from isopropyl, butyl and 2-ethylhexyl.

Preferred acidic organometallic catalysts are, for example, selected from dialkyltin oxides (R¹⁵)₂SnO, where R¹⁵ is as defined above. A particularly preferred representative of acidic organometallic catalysts is di-n-butyltin oxide, which is commercially available as Oxo-tin or as Fascat® grades.

Preferred acidic organic catalysts are acidic organic compounds having, for example, phosphate groups, sulfonic acid groups, sulfate groups or phosphonic acid groups. Particularly preferred sulfonic acids contain at least one sulfonic acid group and at least one saturated or unsaturated, linear, branched and/or cyclic hydrocarbon radical having 1 to 40 carbon atoms and preferably having 3 to 24 carbon atoms. Particular preference is given to aromatic sulfonic acids and specifically alkylaromatic monosulfonic acids having one or more C₁-C₂₈-alkyl radicals and in particular those having C₃-C₂₂-alkyl radicals. Suitable examples are methanesulfonic acid, butanesulfonic acid, benzenesulfonic acid, p-toluenesulfonic acid, xylenesulfonic acid, 2-mesitylenesulfonic acid, 4-ethylbenzenesulfonic acid, isopropylbenzenesulfonic acid, 4-butylbenzenesulfonic acid, 4-octylbenzenesulfonic acid; dodecylbenzenesulfonic acid, didodecylbenzenesulfonic acid, naphthalenesulfonic acid. It is also possible to use acidic ion exchangers as acidic organic catalysts, for example sulfone-group-carrying poly(styrene) resins crosslinked with about 2 mol % of divinylbenzene.

Of particular preference for carrying out the method according to the invention are boric acid, phosphoric acid, polyphosphoric acid and polystyrenesulfonic acids. Particular preference is given to titanates of the general formula Ti(OR¹⁵)₄ and specifically titanium tetrabutylate and titanium tetraisopropylate.

If the use of acidic inorganic, organometallic or organic catalysts is desired, then according to the invention, 0.01 to 10% by weight, preferably 0.02 to 2% by weight, of catalyst is used.

In a further preferred embodiment, the microwave irradiation is carried out in the presence of acidic solid catalysts. Heterogeneous catalysts of this type can be suspended in the reaction mixture and pumped through the reaction tube together with the reaction mixture. In a particularly preferred embodiment, the reaction mixture, optionally admixed with solvent, is passed over a fixed-bed catalyst located in the reaction tube and, in so doing, subjected to microwave radiation. Suitable solid catalysts are, for example, zeolites, silica gel, montmorillonite and (partially) crosslinked polystyrenesulfonic acid, which may optionally be impregnated with catalytically active metal salts. Suitable acidic ion exchangers based on polystyrenesulfonic acids, which can be used as solid-phase catalysts, are obtainable, for example, from Rohm & Haas under the trade name Amberlyst®.

The production according to the invention of the esters takes place by mixing carboxylic acid, alcohol and catalyst and subsequent irradiation of the mixture with microwaves in a reaction tube, the longitudinal axis of which is in the direction of propagation of the microwaves in a monomode microwave applicator.

The irradiation of the reaction mixture with microwaves preferably takes place in a largely microwave-transparent reaction tube located within a hollow conductor connected to a microwave generator. The reaction tube is preferably aligned axially with the central axis of symmetry of the hollow conductor.

The hollow conductor functioning as microwave applicator is preferably configured as a cavity resonator. Further preferably, the microwaves not absorbed in the hollow conductor are reflected at its end. Preferably, the length of the cavity resonator is dimensioned such that a stationary wave is formed therein. By configuring the microwave applicator as a resonator of the reflection type, a local increase in the electrical field strength at the same power supplied by the generator and increased energy exploitation are achieved.

The cavity resonator is preferably operated in the E₀₁ₙ mode, where n is an integer and indicates the number of field maxima of the microwave along the central axis of symmetry of the resonator. In this operation, the electrical field is directed in the direction of the central axis of symmetry of the cavity resonator. It has a maximum in the region of the central axis of symmetry and decreases to the value zero toward the outer surface. This field configuration is rotationally symmetric about the central axis of symmetry. By using a cavity resonator with a length in which n is an integer, the formation of a stationary wave is facilitated. According to the desired flow rate of the reaction material through the reaction tube, the required temperature and the required residence time in the resonator, the length of the resonator is selected relative to the wavelength of the microwave radiation used. Preferably, n is an integer from 1 to 200, particularly preferably from 2 to 100, in particular from 4 to 50, specifically from 3 to 20, such as, for example, three, four, five, six, seven, eight, nine or ten.

The E₀₁ₙ mode of the cavity resonator is also referred to as TM₀₁ₙ mode, see for example K. Lange, K. H. Löcherer, Taschenbuch der Hochfrequenztechnik [Pocket book of high-frequency technology], volume 2, page K21 ff.

The injection of the microwave energy into the hollow conductors functioning as microwave applicator can take place via suitably dimensioned holes or slits. In a particularly preferred embodiment according to the invention, the irradiation of the reaction mixture with microwaves takes place in a reaction tube which is in a hollow conductor with a coaxial transition of the microwaves. Microwave devices particularly preferred for this method are constructed from a cavity resonator, a coupling device for coupling a microwave field into the cavity resonator and with in each case one orifice on two opposite end walls for passage of the reaction tube through the resonator. The microwaves are preferably coupled into the cavity resonator via a coupling pin which projects into the cavity resonator. Preferably, the coupling pin is configured as a preferably metallic inner conductor tube which functions as a coupling antenna. In a particularly preferred embodiment, this coupling pin projects through one of the end orifices into the cavity resonator. The reaction tube particularly preferably adjoins the inner conductor tube of the coaxial transition and is specifically conducted through the cavity thereof into the cavity resonator. The reaction tube is preferably aligned axially with a central axis of symmetry of the cavity resonator. For this, the cavity resonator preferably has in each case one central orifice on two opposite end walls for passage of the reaction tube.

The feeding-in of the microwaves into the coupling pin or into the inner conductor tube functioning as a coupling antenna can take place, for example, by means of a coaxial connecting line. In a preferred embodiment, the microwave field is supplied to the resonator via a hollow conductor, in which case the end of the coupling pin projecting out of the cavity resonator is conducted into an orifice, which is located in the wall of the hollow conductor, into the hollow conductor, and takes microwave energy from the hollow conductor and couples it into the resonator.

In a specific embodiment, the irradiation of the reaction mixture with microwaves takes place in a microwave-transparent reaction tube which is axially symmetrical within a E₀₁ₙ round hollow conductor with a coaxial transition of the microwaves. In this case, the reaction tube is conducted through the cavity of an inner conductor tube functioning as coupling antenna into the cavity resonator. In a further preferred embodiment, the irradiation of the reaction mixture with microwaves takes place in a microwave-transparent reaction tube which is conducted through a E₀₁ₙ cavity resonator with axial feeding-in of the microwaves, where the length of the cavity resonator is dimensioned such that n=2 or more field maxima of the microwave are formed. In a further preferred embodiment, the irradiation of the reaction mixture with microwaves takes place in a microwave-transparent reaction tube which is conducted through a E₀₁ₙ cavity resonator with axial feeding-in of the microwaves, where the length of the cavity resonator is dimensioned such that a stationary wave where n=2 or more field maxima of the microwave is formed. In a further preferred embodiment, the irradiation of the reaction mixture with microwaves takes place in a microwave-transparent reaction tube which is axially symmetric within a circular cylindrical E₀₁ₙ cavity resonator with a coaxial transition of the microwaves, where the length of the cavity resonator is dimensioned such that n=2 or more field maxima of the microwave are formed. In a further preferred embodiment, the irradiation of the reaction mixture with microwaves takes place in a microwave-transparent reaction tube which is axially symmetric within a circular cylindrical E₀₁ₙ cavity resonator with a coaxial transition of the microwaves, where the length of the cavity resonator is dimensioned such that a stationary wave where n=2 or more field maxima of the microwave is formed.

Microwave generators, such as, for example, the magnetron, the klystron and the gyrotron are known to the person skilled in the art.

The reaction tubes used to carry out the method according to the invention are preferably manufactured from largely microwave-transparent, high-melting material. Particular preference is given to using nonmetallic reaction tubes. Largely microwave-transparent is understood here as meaning materials which absorb as little microwave energy as possible and convert it to heat. A measure used for the ability of a substance to absorb microwave energy and convert it to heat is often the dielectric loss factor tan δ=ε″/ε′. The dielectric loss factor tan δ is defined as the ratio of dielectric loss ε″ and dielectric constant ε′. Examples of tan δ values of various materials are given, for example, in D. Bogdal, Microwave-assisted Organic Synthesis, Elsevier 2005. For reaction tubes suitable according to the invention, materials with tan δ values measured at 2.45 GHz and 25° C. of less than 0.01, in particular less than 0.005 and specifically less than 0.001 are preferred. Suitable preferred microwave-transparent and thermally stable materials are primarily mineral-based materials such as, for example, quartz, aluminum oxide, sapphire, zirconium oxide, silicon nitride and the like. Thermally stable plastics such as, in particular, fluoropolymers, such as, for example, Teflon, and industrial plastics such as polypropylene, or polyaryl ether ketones, such as, for example, glass fiber-reinforced polyether ether ketone (PEEK), are also suitable as tube materials. In order to withstand the temperature conditions during the reaction, minerals, such as quartz or aluminum oxide, coated with these plastics have in particular proven to be useful as reactor materials.

Reaction tubes particularly suitable for the method according to the invention have an internal diameter of one millimeter to ca. 50 cm, in particular between 2 mm and 35 cm and specifically between 5 mm and 15 cm, such as, for example, between 10 mm and 7 cm. Reaction tubes are understood here as meaning vessels whose length to diameter ratio is greater than 5, preferably between 10 and 100 000, particularly preferably between 20 and 10 000, such as, for example, between 30 and 1000. The length of the reaction tube is understood here as meaning the length of the reaction tube on which the microwave irradiation takes place. Baffles and/or other mixing elements can be incorporated into the reaction tube.

E₀₁ cavity resonators particularly suitable for the method according to the invention preferably have a diameter which corresponds to at least half the wavelength of the microwave radiation used. Preferably, the diameter of the cavity resonator is 1.0 to 10 times, more preferably 1.1 to 5 times and especially 2.1 to 2.6 times, half the wavelength of the microwave radiation used. Preferably, the E₀₁ cavity resonator has a round cross section, which is also referred to as E₀₁ round hollow conductor. It particularly preferably has a cylindrical shape and specifically a circular cylindrical shape.

The reaction tube is usually provided at the inlet with a metering pump and a manometer, and at the outlet with a pressure-retaining device and a heat exchanger. This makes possible reactions within a very wide pressure and temperature range.

The production of the reaction mixture consisting of carboxylic acid, alcohol and catalyst can be carried out continuously, discontinuously or else in semi-batchwise processes. Thus, the production of the reaction mixture can be carried out in an upstream (semi)-batchwise process, such as, for example, in a stirred vessel. In a preferred embodiment, the starting materials carboxylic acid and alcohol, and also the catalyst, independently of one another optionally diluted with solvent, are only mixed shortly before being introduced into the reaction tube. The catalyst can be added to the reaction mixture as it is or as a mixture with one of the starting materials. For example, it has proven particularly useful to undertake the mixing of carboxylic acid, alcohol and catalyst in a mixing zone, from which the reaction mixture is conveyed into the reaction tube. Further preferably, the starting materials and catalyst are preferably supplied to the method according to the invention in liquid form. For this, it is possible to use relatively high-melting and/or relatively high-viscosity starting materials, for example in the molten state and/or admixed with solvent, for example in the form of a solution, dispersion or emulsion. The catalyst is added to one of the starting materials or else to the starting material mixture prior to entry into the reaction tube. It is also possible to react heterogeneous systems by the process according to the invention, in which case appropriate industrial equipment for conveying the reaction material is required.

The reaction mixture can be fed into the reaction tube either at the end conducted through the inner conductor tube or at the opposite end. The reaction mixture can consequently be conducted through the microwave applicator parallel or anti-parallel to the direction of propagation of the microwaves.

By variation of tube cross section, length of the irradiation zone (this is understood as meaning the length of the reaction tube in which the reaction material is exposed to microwave radiation), flow rate, geometry of the cavity resonator, and the microwave power injected, the reaction conditions are preferably established such that the maximum reaction temperature is achieved as quickly as possible and the residence time at maximum temperature remains sufficiently short that the fewest possible secondary reactions or consecutive reactions occur. To complete the reaction, the reaction material can pass through the reaction tube more than once, optionally after intermediate cooling. In the case of slow reactions, it has often proven useful to keep the reaction product at reaction temperature for a certain time after it leaves the reaction tube. In many cases, it has proven to be useful if the reaction product is cooled immediately after leaving the reaction tube, e.g. by jacket cooling or decompression. It has also proven useful to deactivate the catalyst directly after it has left the reaction tube. This can take place for example by neutralization or, in the case of heterogeneously catalyzed reactions, by filtration.

Preferably, the temperature increase caused by the microwave irradiation is limited to a maximum of 500° C. for example by regulating the microwave intensity, the flow rate and/or by cooling the reaction tube, for example by means of a nitrogen stream. In particular, carrying out the reaction at temperatures between 120° C. and a maximum of 400° C. and specifically between 150° C. and a maximum of 300° C., such as, for example, at temperatures between 180° C. and 270° C., has proven successful.

The duration of the microwave irradiation depends on various factors, such as, for example, the geometry of the reaction tube, the injected microwave energy, the specific reaction and the desired degree of conversion. Usually, the microwave irradiation is undertaken over a period of less than 30 minutes, preferably between 0.01 seconds and 15 minutes, particularly preferably between 0.1 seconds and 10 minutes and in particular between one second and 5 minutes, such as, for example, between 5 seconds and 2 minutes. The intensity (power) of the microwave radiation is adjusted here such that the reaction material has the desired maximum temperature upon leaving the cavity resonator. In a preferred embodiment, the reaction product is cooled as quickly as possible directly after the microwave irradiation is complete to temperatures below 120° C., preferably below 100° C. and especially below 60° C.

Preferably, the reaction is carried out at pressures between 1 bar (atmospheric pressure) and 500 bar, particularly preferably between 1.5 and 200 bar, in particular between 3 bar and 150 bar and especially between 10 bar and 100 bar, such as, for example, between 15 and 50 bar. Working under increased pressure has proven to be particularly useful, which involves working above the boiling temperature (at atmospheric pressure) of the starting materials, products, of any solvent present and/or of the water of reaction formed during the reaction. The pressure is particularly preferably adjusted to a sufficiently high level that the reaction mixture remains in the liquid state and does not boil during the microwave irradiation.

To avoid secondary reactions and to produce the purest possible products, it has proven useful to handle starting materials and products in the presence of an inert protective gas, such as, for example, nitrogen, argon or helium.

Although the starting materials carboxylic acid and alcohol often lead to easy-to-handle reaction mixtures, it has in many cases proven useful to work in the presence of solvents in order, for example, to lower the viscosity of the reaction mixture and/or to fluidize the reaction mixture, especially if it is heterogeneous. For this purpose, it is possible in principle to use all solvents which are inert under the reaction conditions used and do not react with these starting materials and/or the products formed. An important factor when selecting suitable solvents is their polarity, which, on the one hand, determines the dissolution properties and, on the other hand, determines the extent of the interaction with microwave radiation. A particularly important factor when selecting suitable solvents is their dielectric loss ε″. The dielectric loss ε″ describes the proportion of microwave radiation which is converted to heat during the interaction of a substance with microwave radiation. The last-mentioned value has proven to be a particularly important criterion for the suitability of a solvent for carrying out the method according to the invention.

It has proven particularly useful to work in solvents which exhibit the lowest possible microwave absorption and hence make only a small contribution to the heating of the reaction system. Solvents preferred for the method according to the invention have a dielectric loss ε″, measured at room temperature and 2450 MHz, of less than 10 and preferably less than 1, such as, for example, less than 0.5. An overview of the dielectric loss of different solvents can be found, for example, in “Microwave Synthesis” by B. L. Hayes, CEM Publishing 2002. Of suitability for the method according to the invention are in particular solvents with ε″ values below 10, such as N-methylpyrrolidone, N,N-dimethylformamide or acetone, and in particular solvents with ε″ values below 1. Examples of particularly preferred solvents with ε″ values below 1 are aromatic and/or aliphatic hydrocarbons, such as, for example, toluene, xylene, ethylbenzene, tetralin, hexane, cyclohexane, decane, pentadecane, decalin, and also commercial hydrocarbon mixtures, such as benzine fractions, kerosene, solvent naphtha, ®Shellsol AB, Solvesso® 150, Solvesso® 200, Exxsol®, Isopar® and Shellsol® grades. Solvent mixtures which have ε″ values preferably below 10 and specifically below 1 are equally preferred for carrying out the method according to the invention.

In a further preferred embodiment, the method according to the invention is carried out in solvents with higher ε″ values of, for example 5 or higher, such as in particular with ε″ values of 10 and higher. This embodiment has proven to be useful particularly in the case of the reaction of reaction mixtures which themselves, i.e. without the presence of solvents and/or diluents, exhibit only a very low microwave absorption. Thus, this embodiment has proven to be particularly useful in the case of reaction mixtures which have a dielectric loss ε″ of less than 10 and preferably less than 1. However, the accelerated heating of the reaction mixture often observed as a result of the solvent addition requires measures for maintaining the maximum temperature.

When working in the presence of solvents, their proportion in the reaction mixture is preferably between 1 and 95% by weight, particularly preferably between 2 and 90% by weight, specifically between 5 and 85% by weight and in particular between 10 and 75% by weight, such as, for example, between 30 and 60% by weight. The reaction is particularly preferably carried out without a solvent.

In a further preferred embodiment, substances are added to the reaction mixture that are insoluble in said mixture and absorb microwaves to a large extent. These lead to a considerable local heating of the reaction mixture and consequently to further accelerated reactions. One suitable heat collector of this type is, for example, graphite.

Microwaves is the term used to refer to electromagnetic rays with a wavelength between about 1 cm and 1 m and frequencies between about 300 MHz and 30 GHz. This frequency range is suitable in principle for the method according to the invention. For the method according to the invention, preference is given to using microwave radiation with the frequencies approved for industrial, scientific, medical, domestic or similar applications, such as, for example, with frequencies of 915 MHz, 2.45 GHz, 5.8 GHz or 24.12 GHz.

The microwave power to be injected into the cavity resonator for carrying out the method according to the invention is in particular dependent on the desired reaction temperature, but also on the geometry of the reaction tube and hence the reaction volume, and also the flow rate of the reaction material through the heating zone. It is usually between 200 W and several 100 kW and in particular between 500 W and 100 kW, such as, for example, between 1 kW and 70 kW. It can be generated by means of one or more microwave generators.

In a preferred embodiment, the reaction is carried out in a pressure-resistant, chemically inert tube, where the water of reaction which is formed, and possibly starting materials and, if present, solvent, lead to a buildup in pressure. When the reaction is complete, the overpressure can be used by means of decompression for volatalization and removal of water of reaction, excess starting materials, and optionally solvent and/or for cooling the reaction product. In a further embodiment, the water of reaction formed is, after cooling and/or decompression, separated off by customary methods such as, for example, phase separation, distillation, stripping, flashing and/or absorption.

To achieve particularly high degrees of conversion, it has in many cases proven useful to expose the resulting reaction mixture, following removal of water of reaction and also, if appropriate, discharge of product and/or by-product, again to microwave irradiation, in which case the ratio of the reactants used may have to be supplemented to compensate for consumed or deficient starting materials.

The advantages of the method according to the invention are a very uniform irradiation of the reaction material in the center of a symmetric microwave field within a reaction tube, the longitudinal axis of which is in the direction of propagation of the microwaves of a monomode microwave applicator and in particular within a E₀₁ cavity resonator for example with coaxial transition. Here, the reactor design according to the invention also allows reactions to be carried out at very high pressures and/or temperatures. As a result of increasing the temperature and/or pressure, a significant increase in the degree of conversion and yield is observed even compared with known microwave reactors, without resulting in undesired secondary reactions and/or discolorations. Surprisingly, a very high efficiency in the utilization of the microwave energy is achieved here while utilizing the microwave energy injected into the cavity resonator, said efficiency usually being more than 50%, often more than 80%, sometimes more than 90% and in specific cases above 95%, such as, for example, above 98%, of the injected microwave power and hence offers economical and also ecological advantages over conventional production methods and also over microwave methods in the prior art.

Moreover, the method according to the invention allows a controlled, safe and reproducible reaction regime. Since the reaction material is moved in the reaction tube parallel to the direction of propagation of the microwaves, known overheating phenomena as a result of uncontrolled field distributions, which lead to local overheating as a result of changing intensities of the microwave field, for example in wave crests and nodes, are balanced out by the flowing motion of the reaction mixture. The advantages mentioned also make it possible to work with high microwave powers of more than 1 kW, such as, for example, 2 to 10 kW and in particular 5 to 100 kW, and sometimes even higher, and hence, in combination with only a short residence time in the cavity resonator, to accomplish large production quantities of 100 and more tons per year in one plant.

In this connection, it was surprising that, in spite of the only very short residence time of the reaction mixture in the microwave field in the flow tube with continuous flow, very substantial esterification takes place with conversions generally of more than 80%, often even more than 90%, such as, for example, more than 95%, based on the component used in deficit, without the formation of noteworthy amounts of by-products. Furthermore, it was surprising that the stated conversions can be achieved under these reaction conditions without separating off the water of reaction formed during the esterification. In the case of a corresponding reaction of these reaction mixtures in a flow tube with identical dimensions and with thermal jacket heating, to achieve suitable reaction temperatures, extremely high wall temperatures are required, which led to the formation of undefined polymers and colored species, but bring about significantly lower ester formation in the same time interval. Furthermore, the products produced by the method according to the invention have very low metal contents, without requiring further work-up of the crude products. For example, the metal contents of the products produced by the method according to the invention, based on iron as the main element, are usually below 25 ppm, preferably below 15 ppm, specifically below 10 ppm, such as, for example, between 0.01 and 5 ppm, of iron.

The method according to the invention thus allows a very rapid, energy-saving and cost-effective production of carboxylic acid esters in high yields and with high purity in industrial-scale amounts. Besides water of reaction, this method does not produce any significant amounts of by-products. Such rapid and selective reactions cannot be achieved by conventional methods and were not to be expected solely as a result of heating to high temperatures.

### EXAMPLES

The reactions of the reaction mixtures under microwave irradiation were carried out in a ceramic tube (60×1 cm) which was located in axial symmetry in a cylindrical cavity resonator (60×10 cm). On one of the ends of the cavity resonator, the ceramic tube passed through the cavity of an inner conductor tube functioning as coupling antenna. The microwave field with a frequency of 2.45 GHz, produced by a magnetron, was coupled into the cavity resonator by means of the coupling antenna (E₀₁ cavity applicator; monomode), in which a stationary wave was formed.

The microwave power was in each case adjusted via the experiment time in such a way that the desired temperature of the reaction material at the end of the irradiation zone was kept constant. The microwave powers specified in the experiment descriptions therefore represent the mean value of the injected microwave power over time. The measurement of the temperature of the reaction mixture was undertaken directly after it had left the reaction zone (distance of about 15 cm in an insulated stainless steel capillary, Ø 1 cm) by means of a Pt100 temperature sensor. Microwave energy not absorbed directly by the reaction mixture was reflected at the end of the cavity resonator positioned at the opposite end to the coupling antenna; the microwave energy which was also not absorbed by the reaction mixture on the return path and reflected back in the direction of the magnetron was passed with the aid of a prism system (circulator) into a water-containing vessel. The difference between energy injected and heating of this water load was used to calculate the microwave energy introduced into the reaction material.

By means of a high-pressure pump and of a suitable pressure-release valve, the reaction mixture in the reaction tube was placed under a operating pressure which sufficed to always keep all of the starting materials and products or condensation products in the liquid state. The reaction mixtures produced from carboxylic acid and alcohol were pumped at a constant flow rate through the reaction tube, and the residence time in the irradiation zone was adjusted by modifying the flow rate.

The products were analyzed by means of ¹H-NMR spectroscopy at 500 MHz in CDCl₃. The properties were determined by means of atomic absorption spectroscopy.

### Example 1

### Production of Methyl Benzoate

In a 10 l Büchi stirred autoclave with stirrer, internal thermometer and pressure equalizer, 2.36 kg of methanol (73.5 mol) were introduced as initial charge and admixed with 3 kg of benzoic acid (24.5 mol) and 50 g of methanesulfonic acid.

The mixture obtained in this way was pumped through the reaction tube continuously at 5 l/h at an operating pressure of 35 bar and exposed to a microwave power of 2.0 kW, 96% of which was absorbed by the reaction material. The residence time of the reaction mixture in the irradiation zone was ca. 34 seconds. At the end of the reaction tube, the reaction mixture had a temperature of 260° C. The reaction mixture was cooled to room temperature directly after leaving the reactor using a high-intensity heat exchanger.

A conversion of 85% of theory was achieved. The reaction product was virtually colorless and comprised <1 ppm of iron. Following neutralization of the catalyst with hydrogencarbonate solution and distillative removal of water and excess methanol, 2.71 kg of methyl benzoate with a purity of >98% were obtained by means of vacuum distillation.

### Example 2

### Production of Butyl p-Toluate

In a 10 l Büchi stirred autoclave with stirrer, internal thermometer and pressure equalizer, 3.12 kg of n-butanol (42 mol) were introduced as initial charge and admixed with 2.88 kg of p-toluic acid (21 mol) and 60 g of methanesulfonic acid.

The mixture obtained in this way was pumped through the reaction tube continuously at 3 l/h at an operating pressure of 25 bar and exposed to a microwave power of 3.2 kW, 92% of which was absorbed by the reaction material. The residence time of the reaction mixture in the irradiation zone was ca. 57 seconds. At the end of the reaction tube, the reaction mixture had a temperature of 272° C. the reaction mixture was cooled to room temperature directly after leaving the reactor using a high-intensity heat exchanger.

A conversion of 82% of theory was achieved. The reaction product was virtually colorless and comprised <5 ppm of iron. Following neutralization of the catalyst with hydrogencarbonate solution and distillative removal of water and excess butanol, 3.3 kg of butyl toluate with a purity of >97.5% were obtained by means of vacuum distillation.

### Example 3

### Production of Methyl p-Hydroxybenzoate

In a 10 l Büchi stirred autoclave with stirrer, internal thermometer and pressure equalizer, 2.7 kg of methanol (84 mol) were introduced as initial charge and, with heating to 60° C., 3.8 kg of p-hydroxybenzoic acid (27.5 mol) and 65 g of sulfuric acid (96% strength) were dissolved therein.

The homogeneous solution obtained in this way was pumped continuously through the reaction tube at 6.2 l/h at an operating pressure of 38 bar and exposed to a microwave power of 2.8 kW, 94% of which was absorbed by the reaction material. The residence time of the reaction mixture in the irradiation zone was ca. 27 seconds. At the end of the reaction tube, the reaction mixture had a temperature of 267° C. The reaction mixture was cooled to room temperature directly after leaving the reactor using a high-intensity heat exchanger.

A conversion of 87% of theory was achieved. The reaction product was virtually colorless and comprised <2 ppm of iron. Following neutralization of the catalyst with ammonium hydrogencarbonate solution and distillative removal of water and excess methanol, 3.6 kg of crude methyl p-hydroxybenzoate in a mixture with ammonium sulfate and unreacted starting material were obtained. By means of recrystallization from methanol, the product was isolated with a purity of >99%.

### Example 4

### Production of Oligomeric PET

In a 10 l Büchi stirred autoclave with stirrer, internal thermometer and pressure equalizer, 1.86 kg of ethylene glycol (30 mol) were introduced as initial charge and heated to 120° C., and 4.14 kg of terephthalic acid (25 mol) were dissolved therein.

The homogeneous solution obtained in this way was pumped continuously through the reaction tube at 6 l/h at an operating pressure of 30 bar and subjected to a microwave power of 3 kW, 92% of which was absorbed by the reaction material. The residence time of the reaction mixture in the irradiation zone was ca. 28 seconds. At the end of the reaction tube, the reaction mixture had a temperature of 255° C. The reaction mixture was cooled to room temperature directly after leaving the reactor using a high-intensity heat exchanger.

The reaction product was considerably viscous and virtually colorless. It comprised <5 ppm of iron.

Before the reaction, an acid number of 471 (mg of KOH/g of sample) was determined by means of titration. After the reaction, the value was 52. Accordingly, the conversion was about 89% based on the COOH functionalities used. According to the statistics of the polycondensation, the average chain length of the oligomers was thus 8-10 PET units or approximately 1700 g/mol.

### Example 5

### Production of Dimethyl 2,6-Pyridinedicarboxylate

In a 10 l Büchi stirred autoclave with stirrer, internal thermometer and pressure equalizer, 3.9 kg of methanol (120 mol) were introduced as initial charge and admixed with 2 kg of 2,6-pyridinedicarboxylic acid (12 mol) and 60 g of methanesulfonic acid and dissolved in one another by gentle heating to 60° C.

The mixture obtained in this way was pumped continuously through the reaction tube at 4 l/h at an operating pressure of 35 bar and exposed to a microwave power of 2.4 kW, 93% of which was absorbed by the reaction material. The residence time of the reaction mixture in the irradiation zone was ca. 42 seconds. At the end of the reaction tube, the reaction mixture had a temperature of 240° C. The reaction mixture was cooled to room temperature directly after leaving the reactor using a high-intensity heat exchanger.

A conversion of 83% of theory was achieved. The reaction product was virtually colorless and comprised <5 ppm of iron. Following neutralization of the catalyst with hydrogencarbonate solution and distillative removal of water and excess methanol, 1.8 kg of dimethyl 2,6-pyridinedicarboxylate with a purity of >99% were obtained by distillation.

1. A continuous method for producing aromatic carboxylic esters, in which at least one aromatic carboxylic acid of the formula (I)
Ar—COOH (I)
in which Ar is an optionally substituted aryl radical having 5 to 50 atoms, is reacted with at least one alcohol of the formula (II)
R²—(OH)ₙ (II)
in which
R² is an optionally substituted hydrocarbon radical having 1 to 100 carbon atoms and
n is a number from 1 to 10,
in the presence of at least one esterification catalyst with microwave irradiation in a reaction tube, the longitudinal axis of which extends in the direction of propagation of the microwaves of a monomode microwave applicator, to give the ester.

2. The method as claimed in claim 1, in which the irradiation of the reaction mixture takes place with microwaves in a largely microwave-transparent reaction tube within a hollow conductor connected via waveguides to a microwave generator.

3. The method as claimed in one or more of claims 1 and 2, in which the microwave applicator is designed as a cavity resonator.

4. The method as claimed in one or more of claims 1 to 3, in which the microwave applicator is configured as a cavity resonator of the reflection type.

5. The method as claimed in one or more of claims 1 to 4, in which the reaction tube is aligned axially with a central axis of symmetry of the hollow conductor.

6. The method as claimed in one or more of claims 1 to 5, in which the irradiation of the reaction mixture takes place in a cavity resonator with a coaxial transition of the microwaves.

7. The method as claimed in one or more of claims 1 to 6, in which the cavity resonator is operated in the E₀₁ₙ mode, where n is an integer from 1 to 200.

8. The method as claimed in one or more of claims 1 to 7, in which a stationary wave is formed in the cavity resonator.

9. The method as claimed in one or more of claims 1 to 8, in which the reaction material is heated by the microwave irradiation to temperatures between 150 and 500° C.

10. The method as claimed in one or more of claims 1 to 9, in which the microwave irradiation takes place at pressures above atmospheric pressure.

11. The method as claimed in one or more of claims 1 to 10, in which Ar is a cyclic, through-conjugated system with (4n+2) π electrons, in which n is 1, 2, 3, 4 or 5.

12. The method as claimed in one or more of claims 1 to 11, in which Ar is an alkylaryl group with at least one alkyl radical having 1 to 50 carbon atoms.

13. The method as claimed in one or more of claims 1 to 12, in which Ar carries two or more hydroxyl groups and/or hydroxyalkyl groups.

14. The method as claimed in one or more of claims 1 to 11, in which Ar is selected from benzoic acid, phthalic acid, isophthalic acid, terephthalic acid, the isomers of naphthalenecarboxylic acid, of pyridinecarboxylic acid and of naphthalenedicarboxylic acid, trimellitic acid, trimesic acid, pyromellitic acid and mellitic acid, the isomers of methoxybenzoic acid, hydroxybenzoic acid, hydroxymethylbenzoic acid, hydroxymethoxybenzoic acid, hydroxydimethoxybenzoic acid, hydroxyisophthalic acid, hydroxynaphthalenecarboxylic acid, hydroxypyridinecarboxylic acid, hydroxymethylpyridinecarboxylic acid, hydroxyquinolinecarboxylic acid, o-toluic acid, m-toluic acid, p-toluic acid, o-ethylbenzoic acid, m-ethylbenzoic acid, p-ethylbenzoic acid, o-propylbenzoic acid, m-propylbenzoic acid, p-propylbenzoic acid, 3,4-dimethylbenzoic acid, and thiophenecarboxylic acid.

15. The method as claimed in one or more of claims 1 to 14, in which R² is an optionally substituted aliphatic radical having 2 to 24 carbon atoms.

16. The method as claimed in one or more of claims 1 to 14, in which R² is an optionally substituted C₆-C₁₂-aryl group or an optionally substituted heteroaromatic group having 5 to 12 ring members.

17. The method as claimed in one or more of claims 1 to 16, in which n is one, two, three, four, five or six.

18. The method as claimed in one or more of claims 1 to 14, in which R² is radicals of the formula (III)
—(R⁴—O)ₙ—R⁵ (III)
in which
R⁴ is an alkylene group having 2 to 18 carbon atoms or mixtures thereof,
R⁵ is hydrogen or a hydrocarbon radical having 1 to 24 carbon atoms or a group of the formula —R⁴—NR¹⁰R¹¹,
n is a number between 1 and 500 and
R¹⁰, R¹¹ independently of one another, are an aliphatic radical having 1 to 24 carbon atoms, an aryl group or heteroaryl group having 5 to 12 ring members, a poly(oxyalkylene) group having 1 to 50 poly(oxyalkylene) units, where the polyoxyalkylene units are derived from alkylene oxide units having 2 to 6 carbon atoms, or R¹⁰ and R¹¹ together with the nitrogen atom to which they are bonded form a ring having 4, 5, 6 or more ring members.

19. The method as claimed in one or more of claims 1 to 18, in which aromatic carboxylic acid (I) and alcohol (II) are reacted in the molar ratio from 20:1 to 1:20, in each case based on the mole equivalents of carboxyl and hydroxyl groups.

20. The method as claimed in one or more of claims 1 to 19, which is carried out in the presence of homogeneous catalysts, heterogeneous catalysts or mixtures thereof.
